(19) United States
(12) Patent Application Publication
(10) Pub. No.: US 2014/0275102 A1
(43) Pub. Date: Sep. 18, 2014

(54) Solid State Forms of 6-[4-[3-((R)-2-Methylpyrrolidine-1-yl)-propoxy]phenyl] 2H-pyridazine-3-one Hydrochloride

(71) Applicant: Cephalon Inc., Frazer, PA (US)

(72) Inventors: Stephen J. Bierlmaier, Thorndale, PA (US); Laurent Courvoisier, (US); Ralph C. Haltiwanger, West Chester, PA (US); Martin J. Jacobs, Versailles, KY (US)

(73) Assignee: CEPHALON, INC., Frazer, PA (US)

(21) Appl. No.: 14/207,465

(22) Filed: Mar. 12, 2014

Related U.S. Application Data
(60) Provisional application No. 61/782,216, filed on Mar. 14, 2013.

(51) Int. Cl. C07D 403/12

(52) U.S. Cl. CPC C07D 403/12
USPC 514/252.05; 544/238

(57) ABSTRACT

Solid state forms of the compound 6-[4-[3-((R)-2-methylpyrrolidine-1-yl)-propoxy]phenyl]2H-pyridazine-3-one hydrochloride (Compound 1), processes for preparing the solid state forms, and pharmaceutical compositions thereof, are provided. Compound 1 is a histamine H3 receptor antagonist/inverse agonist. Thus the provided solid state forms are useful, for example, for the manufacture of a medicament for the treatment of disorders mediated by the H3 receptor.

[chemical structure]

### CROSS REFERENCE TO RELATED APPLICATIONS

This application claims the benefit of U.S. Provisional Application No. 61/782,216, filed Mar. 14, 2013, the contents of which is hereby incorporated by reference in its entirety.

Throughout this application, various U.S. patents and patent application publications are referenced. The disclosures of these documents are hereby incorporated by reference into this application to describe more fully the art to which this invention pertains.

### FIELD OF THE INVENTION

The present invention provides solid state forms of the compound 6-[4-[3-((R)-2-methylpyrrolidine-1-yl)-propoxy]phenyl]2H-pyridazine-3-one hydrochloride and pharmaceutical compositions comprising these solid state forms.

### BACKGROUND OF THE INVENTION

The compound 6-[4-[3-((R)-2-methylpyrrolidine-1-yl)-propoxy]phenyl]-2H-pyridazine-3-one hydrochloride (referred to herein as Compound 1) is a histamine H-3 receptor antagonist/inverse agonist. Possible variations in the nomenclature for the naming of Compound 1 can include, for example, (R)-6-(4-(3-(2-methylpyrrolidin-1-yl)propoxy)phenyl)pyridazin-3(2H)-one hydrochloride. The structure of Compound 1 is provided below:

[chemical structure]

Compound 1 is described in U.S. Pat. Nos. 8,207,168 and 8,247,414, and also in US patent application publications US20110288075 and US 20100273779. The present invention relates to solid state forms of Compound 1.

Polymorphism, the occurrence of different crystal forms, is a property of some molecules and molecular complexes. A single molecule may give rise to a variety of polymorphs having distinct crystal structures and physical properties. These varying physical properties like melting point and thermal behaviors. Analytical methods employed to characterize solid state forms include, for example thermogravimetric analysis (TGA), differential scanning calorimetry (DSC), X-ray powder diffraction (XRPD), infrared (IR) (or Fourier Transform infrared (FTIR)), and Raman spectroscopy, Gravimetric Vapor Sorption (GVS), and solid state nuclear magnetic resonance (ssNMR). One or more of these analytical methods may be used to distinguish different polymorphic forms of a compound.

Different solid state forms of an active pharmaceutical ingredient may possess different properties. Such variations in the properties of different solid state forms may provide bases for improving formulation, for example, by facilitating better processing or handling characteristics, improving the dissolution profile, or improving stability and shelf-life. These variations in the properties of different solid state forms may also provide improvements to the final dosage form, for instance, if they serve to improve bioavailability. Different crystalline forms often provide opportunities to assess variations in the properties and characteristics of a solid active pharmaceutical ingredient.

Discovering different solid state forms of an active pharmaceutical ingredient can provide materials having desirable processing properties, such as ease of handling, ease of processing, storage stability, ease of purification, or as desirable intermediate crystal forms that facilitate conversion to other polymorphic forms. Different solid state forms of a pharmaceutically active compound can also provide an opportunity to improve the performance characteristics of a pharmaceutical product containing that compound. Discovering different solid state forms can also serve to enlarge the repertoire of materials that a formulation scientist has available for formulation optimization, for example by providing products with varying properties, e.g., better processing characteristics or handling characteristics, or improved shelf-life.

### SUMMARY OF THE INVENTION

The present invention provides solid state forms of 6-[4-[3-((R)-2-methylpyrrolidine-1-yl)-propoxy]phenyl]2H-pyridazine-3-one hydrochloride (Compound 1), e.g., crystalline polymorphs designated herein as Form A1, Form B1 and Form H4A1. The invention also provides pharmaceutical compositions comprising the solid state forms described herein, and at least one pharmaceutically acceptable excipient.

The present invention also encompasses the solid state forms described herein for use as medicaments, particularly for the treatment of a disorder mediated by histamine, more particularly a disorder mediated by the histamine H3 receptor and treatable by an agent having antagonist activity at the H3 histamine receptor. Such disorders include, for example, narcolepsy or sleep/wake disorders, feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders, epilepsy, gastrointestinal disorders, respiratory disorders, inflammation, and myocardial infarction.

The present invention further provides a pharmaceutical composition comprising any one of the solid state forms provided herein, and at least one pharmaceutically acceptable excipient, for use as medicaments, particularly for the treatment of disorders as described above. Processes for preparing the above pharmaceutical compositions are also provided.

The present invention also provides a method of treating a disorder mediated by the histamine H3 receptor and treatable by an agent having antagonist activity at the H3 histamine receptor. The method comprises administering a therapeutically effective amount of at least one of the solid state forms of the present invention, or a pharmaceutical composition comprising at least one of the solid state forms, to a person suffering from such a disorder or in need of such a treatment. Disorders treatable by this method include, for example, narcolepsy or sleep/wake disorders, feeding behavior, eating disorders, obesity, cognition, arousal, memory, mood disorders, mood attention alteration, attention deficit hyperactivity disorder (ADHD), Alzheimer's disease/dementia, schizophrenia, pain, stress, migraine, motion sickness, depression, psychiatric disorders, epilepsy, gastrointestinal disorders, respiratory disorders, inflammation, and myocardial infarction.

### BRIEF DESCRIPTION OF THE DRAWINGS

FIG. 1 shows an XRPD pattern of Form A1 of Compound 1.

FIG. 2 shows an overlay depicting variable temperature X-ray powder diffraction (VT-XRPD) Patterns of Form A1 of Compound 1.

FIG. 3 shows a overlay of DSC and TGA curves for Form A1 of Compound 1.

FIG. 4 shows a GVS Isotherm plot for Form A1 of Compound 1.

FIG. 5 shows XRPD Diffractograms of Form A1 before and after GVS Analysis.

FIG. 6 shows an FTIR spectrum of Form A1 of Compound 1.

FIG. 7 shows a Raman spectrum of Form A1 of Compound 1.

FIG. 8 shows an XRPD pattern for Form B1 of Compound 1.

FIG. 9 shows an overlay depicting variable temperature XRPD Patterns of Form B1.

FIG. 10 shows a overlay of DSC and TGA curves of Form B1 of Compound 1.

FIG. 11 shows an XRPD pattern of Form H4A1 of Compound 1.

FIG. 12 shows a overlay of DSC and TGA curves for Form H4A1 of Compound 1.

FIG. 13 shows a GVS Isotherm plot for Form H4A1 of Compound 1.

FIG. 14 shows XRPD diffractograms of Form H4A1 before and after GVS.

FIG. 15 shows an FTIR spectrum for Form H4A1

FIG. 16 shows a Raman spectrum for Form H4A1

FIG. 17 shows an overlay of observed versus calculated XRPD data for Form A1.

FIG. 18 shows the structure of Compound 1 from the single crystal structure of Form A1.

FIGS. 19-21 show three views of the molecular packing for Form A1.

FIG. 22 shows an overlay of X-ray powder diffractograms assessing the stress stability of A1 to grinding as a function of time.

FIG. 23 shows an overlay of X-ray powder diffractograms assessing the stress stability of H4A1 to grinding as a function of time.

FIG. 24 shows an overlay of XRPD analyses of certain amorphous crystallization products prepared in Example 1(d).

### DETAILED DESCRIPTION OF THE INVENTION

The present invention provides solid state forms of 6-[4-[3-((R)-2-methylpyrrolidine-1-yl)-propoxy]phenyl]2H-pyridazine-3-one hydrochloride (Compound 1). The solid state forms include three crystalline polymorphs.

According to some embodiments, the solid state forms according to the invention are substantially free of any other solid state forms of Compound 1. In any embodiment of the present invention, by “substantially free” is meant that the solid state forms of the invention contain 20% (w/w) or less, 10% (w/w) or less, 5% (w/w) or less, 2% (w/w) or less, 1% (w/w) or less, or 0.5% (w/w) or less of any other solid state forms of Compound 1.

The solid state forms provided herein have advantageous properties selected from at least one of: chemical purity, flowability, solubility, dissolution rate, morphology or crystal habit, stability—such as thermal and mechanical stability to polymorphic conversion, stability to dehydration and/or storage stability, low content of residual solvent, a lower degree of hygroscopicity, flowability, and advantageous processing and handling characteristics such as compressibility, and bulk density.

A crystal form may be referred to herein as being characterized by graphical data “as depicted in” a Figure. Such data include, for example, powder X-ray diffractograms. The skilled person will understand that such graphical representations of data may be subject to small variations, e.g., in peak relative intensities and peak positions due to factors such as variations in instrument response and variations in sample concentration and purity, which variations are well known to the skilled person. Accordingly, the skilled person would readily be capable of comparing the graphical data in the Figures herein with graphical data generated for an unknown crystal form and confirm whether the two sets of graphical data are characterizing the same crystal form or two different crystal forms. A crystal form of Compound 1 referred to herein as being characterized by graphical data “as depicted in” a Figure will thus be understood to include any crystal forms of Compound 1 characterized with graphical data having such small variations, as are well known to the skilled person, in comparison with the Figure.

As used herein, the term “isolated” in reference to any of solid state forms of the invention corresponds to a solid state form of Compound 1 that is physically separated from the mixture in which it is formed.

The term “solid state form” as used herein, refers to both crystalline and amorphous (non-crystalline) forms of Compound I and mixtures thereof in any ratio. It should be understood that the term solid state form includes crystalline and amorphous (non-crystalline) hydrates and solvates of Compound I as well.

According to one embodiment, the invention comprises a crystalline form of Compound 1, designated as Form A1. Form A1 of Compound 1 can be characterized by an X-ray powder diffraction pattern having peaks at 3.75, 10.98, 14.62, 15.25 and 15.88 degrees two theta±0.2 degrees two theta. Form A1 of Compound 1, as characterized above by X-ray powder diffraction peaks at 3.75, 10.98, 14.62, 15.25 and 15.88 degrees two theta±0.2 degrees two theta, can be further characterized by one or more additional X-ray powder diffraction peaks selected from 16.48, 16.64, 17.19, 18.26 and 20.63 degrees two theta±0.2 degrees two theta.

Alternatively, Form A1 of Compound 1 can be characterized by an X-ray powder diffraction pattern having any selection of from five to ten peaks selected from 3.75, 10.98, 14.62, 15.25, 15.55, 15.88, 16.48, 16.64, 17.19, 18.26, 20.63, 21.08, 21.67, 23.02, 23.29, 23.56, 24.43, 25.78, 26.07, 26.28, 26.33, 27.42, 27.95, 28.40, 29.35, and 29.77 degrees two theta±0.2 degrees two theta.

Form A1 of Compound 1, as characterized by any of the above sets of powder X-ray diffraction data, can optionally be further characterized by additional data selected from one or more of: a powder X-ray diffraction pattern as depicted in FIG. 1, a DSC curve having an endotherm with an onset at 239.5 degrees C. (ΔH 113.9 J/g), a DSC curve as depicted in FIG. 2, A TGA curve as depicted in FIG. 2, an FTIR spectrum as depicted in FIG. 6, and a Raman spectrum as depicted in FIG. 7.

Alternatively, Form A1 of Compound 1 can be characterized by a single crystal structure in a C2 space group with unit cell dimensions of: a=10.8386(10) Å, b=6.9192(5) Å, c=24.432(3) Å, α=γ=90°, β=95.092(9)° and Volume=1825.0(3) Å³, or by the X-ray crystal structure as depicted in FIG. 18, 19, 20 or 21.

Table 1 below lists the most prominent peaks in the X-ray powder diffraction pattern of Form A1 that are provided in FIG. 1; providing the two theta positions (2θ), the D-spacings and the Relative Intensities of the peaks that are listed.

TABLE 1

| | Pos. | d-spacing | Rel. Int. |
|---|---|---|---|
| No. | [2θ] | [Å] | [%] |
| 1 | 3.75 | 23.56 | 21.23 |
| 2 | 10.98 | 8.05 | 16.24 |
| 3 | 14.62 | 6.06 | 41.90 |
| 4 | 15.25 | 5.80 | 63.73 |
| 5 | 15.55 | 5.69 | 69.59 |
| 6 | 15.88 | 5.58 | 30.35 |
| 7 | 16.48 | 5.37 | 17.56 |
| 8 | 16.64 | 5.32 | 23.08 |
| 9 | 17.19 | 5.15 | 23.81 |
| 10 | 18.26 | 4.86 | 30.70 |
| 11 | 20.63 | 4.30 | 14.56 |
| 12 | 21.08 | 4.21 | 17.69 |
| 13 | 21.67 | 4.10 | 20.11 |
| 14 | 23.02 | 3.86 | 7.26 |
| 15 | 23.29 | 3.82 | 22.75 |
| 16 | 23.56 | 3.77 | 35.11 |
| 17 | 24.43 | 3.64 | 7.69 |
| 18 | 25.78 | 3.45 | 31.59 |
| 19 | 26.07 | 3.42 | 38.89 |
| 20 | 26.28 | 3.39 | 100 |
| 21 | 26.33 | 3.38 | 96.27 |
| 22 | 27.42 | 3.25 | 5.76 |
| 23 | 27.95 | 3.19 | 8.97 |
| 24 | 28.40 | 3.14 | 12.21 |
| 25 | 29.35 | 3.04 | 10.84 |
| 26 | 29.77 | 3.00 | 5.64 |

Form A1 of Compound 1 demonstrates stability on storage. No significant changes were observed during 4 weeks of storage at 40 degrees C. at 75% relative humidity as assessed by XRPD.

According to another embodiment, the invention comprises a crystalline form of Compound 1, designated as Form H4A1. Form H4A1 comprises a hydrated form of Compound 1. Form H4A1 is believed to comprise a tetrahydrate form of Compound 1. According to some embodiments of the invention, Form H4A1 of Compound 1 comprises from about 15 to about 20 wt. % of water. According to some embodiments of the invention, Form H4A1 of Compound 1 comprises from about 16 to about 18 wt. % of water. According to some embodiments of the invention, Form H4A1 of Compound 1 comprises from about 17 to about 17.5 wt. % of water.

Form H4A1 of Compound 1 can be characterized by an X-ray powder diffraction pattern having peaks at 5.72, 11.40, 12.95, 16.45 and 17.11 degrees two theta±0.2 degrees two theta. Form H4A1 of Compound 1, as characterized above by X-ray powder diffraction peaks at 5.72, 11.40, 12.95, 16.45 and 17.11 degrees two theta±0.2 degrees two theta, can be further characterized by one or more additional X-ray powder diffraction peaks selected from 17.34, 21.45, and 22.26 degrees two theta±0.2 degrees two theta.

Alternatively, Form H4A1 of Compound 1 can be characterized by an X-ray powder diffraction pattern having any selection of from five to eight peaks selected from 5.72, 11.40, 12.95, 16.45, 17.11, 17.34, 21.45, and 22.26 degrees two theta±0.2 degrees two theta.

Form H4A1 of Compound 1, as characterized by any of the above sets of powder X-ray diffraction data, can optionally be further characterized by additional data selected from one or more of: a powder X-ray diffraction pattern as depicted in FIG. 11, a broad endotherm at 58° C., a DSC curve as depicted in FIG. 12, a TGA weight loss (weight %) over the temperature range of 25-150° C. of 162%, a TGA curve as depicted in FIG. 12, an FTIR spectrum as depicted in FIG. 15, and a Raman spectrum as depicted in FIG. 16.

Table 2 below lists the most prominent peaks in the diffraction pattern of Form H4A1 that is provided in FIG. 11, providing the two theta positions (2θ), the D-spacings and the Relative Intensities of the peaks that are listed.

TABLE 2
X-ray powder diffraction Peak Table for Form H4A1.

| | Pos. | d-spacing | Rel. Int. |
|---|---|---|---|
| No. | [2θ] | [Å] | [%] |
| 1 | 5.72 | 15.44 | 60.2 |
| 2 | 9.77 | 9.04 | 0.7 |
| 3 | 11.40 | 7.76 | 6.9 |
| 4 | 11.56 | 7.61 | 2.1 |
| 5 | 12.10 | 7.31 | 0.1 |
| 6 | 12.95 | 6.83 | 6.1 |
| 7 | 14.34 | 6.17 | 1.5 |
| 8 | 14.45 | 6.12 | 1.6 |
| 9 | 15.29 | 5.79 | 0.3 |
| 10 | 15.61 | 5.67 | 0.5 |
| 11 | 16.45 | 5.39 | 100.0 |
| 12 | 17.11 | 5.18 | 36.0 |
| 13 | 17.34 | 5.11 | 12.0 |
| 14 | 17.66 | 5.02 | 0.7 |
| 15 | 18.62 | 4.76 | 1.0 |
| 16 | 18.84 | 4.71 | 1.2 |
| 17 | 19.12 | 4.64 | 0.8 |
| 18 | 19.38 | 4.58 | 0.4 |
| 19 | 19.55 | 4.54 | 0.4 |
| 20 | 19.86 | 4.47 | 0.3 |
| 21 | 20.75 | 4.28 | 0.4 |
| 22 | 21.45 | 4.14 | 25.4 |
| 23 | 21.89 | 4.06 | 0.4 |
| 24 | 22.26 | 3.99 | 5.4 |
| 25 | 22.85 | 3.89 | 0.5 |
| 26 | 24.18 | 3.68 | 1.7 |
| 27 | 24.31 | 3.66 | 2.3 |
| 28 | 24.66 | 3.61 | 0.8 |
| 29 | 25.16 | 3.54 | 3.0 |
| 30 | 25.32 | 3.52 | 3.0 |
| 31 | 25.71 | 3.46 | 2.4 |

According to another embodiment, the invention comprises a crystalline form of Compound 1, designated as Form B1. Form B1 of Compound 1 can be characterized by an X-ray powder diffraction pattern having peaks at 6.87, 13.79, 15.76, 19.25 and 25.79 degrees two theta±0.2 degrees two theta. Form B1 of Compound 1, as characterized above by X-ray powder diffraction peaks at 6.87, 13.79, 15.76, 19.25 and 25.79 degrees two theta±0.2 degrees two theta, can be further characterized by additional data selected from one or more of: a powder X-ray diffraction pattern as depicted in FIG. 8, a DSC curve as depicted in FIG. 10, and a TGA curve as depicted in FIG. 10.

Table 2 below lists the most prominent peaks in the diffraction pattern of Form B1 that is provided in FIG. 8, providing the two theta positions (2θ), the D-spacings and the Relative Intensities of the peaks that are listed.

TABLE 3
XRPD Peak Table for Form B1.

| | Pos. | d-spacing | Rel. Int. |
|---|---|---|---|
| No. | [2θ] | [Å] | [%] |
| 1 | 4.71 | 18.73 | 7 |
| 2 | 6.87 | 12.86 | 44 |
| 3 | 8.88 | 9.95 | 6 |
| 4 | 10.79 | 8.20 | 10 |
| 5 | 12.10 | 7.31 | 4 |
| 6 | 13.35 | 6.63 | 17 |
| 7 | 13.79 | 6.42 | 15 |
| 8 | 14.56 | 6.08 | 5 |
| 9 | 15.76 | 5.62 | 100 |
| 10 | 17.04 | 5.20 | 17 |
| 11 | 17.88 | 4.96 | 12 |
| 12 | 19.25 | 4.61 | 37 |
| 13 | 21.65 | 4.10 | 16 |
| 14 | 23.08 | 3.85 | 5 |
| 15 | 24.42 | 3.64 | 9 |
| 16 | 25.79 | 3.45 | 68 |
| 17 | 27.13 | 3.28 | 9 |
| 18 | 27.78 | 3.21 | 7 |
| 19 | 28.57 | 3.12 | 4 |
| 20 | 29.31 | 3.055 | 3 |

Having thus described the invention with reference to particular preferred embodiments and illustrative examples, those in the art can appreciate modifications to the invention as described and illustrated that do not depart from the spirit and scope of the invention as disclosed in the specification. The Examples below are set forth to aid in understanding the invention but are not intended to, and should not be construed to limit its scope in any way.

### EXAMPLES

### I. X-Ray Powder Diffraction

Powder X-Ray Diffraction patterns were recorded on a PANalytical X Pert Pro diffractometer equipped with an X celerator detector using Cu Kα radiation at 40 kV and 40 mA. Kα1 radiation is obtained with a highly oriented crystal (Ge111) incident beam monochromator. A 10 mm beam mask, and fixed (¼°) divergence and anti-scatter (⅛°) slits were inserted on the incident beam side. A fixed 0.10 mm receiving slit was inserted on the diffracted beam side. The X-ray powder pattern scan was collected from ca. 2 to 40° 2θ with a 0.0080° step size and 96.06 sec counting time which resulted in a scan rate of approximately 0.5°/min. The sample was spread on silicon zero background (ZBG) plate for the measurement. The sample was rotated at 4°/min on a PANalytical PW3064 Spinner. Measurement of the Si reference standard before the data collection resulted in values for 2θ and intensity that were well within the tolerances of 28.42<2θ<28.50 and significantly greater than the minimum peak height of 150 cps.

### II. Variable Temperature X-Ray Powder Diffraction (VT-XRPD)

Variable temperature studies were performed with an Anton Paar TTK450 temperature chamber under computer control through an Anton Paar TCU100 temperature control unit. Typically the measurements were done with a nitrogen flow through the camera. Two measurement schemes were used, restricted and continuous. In the restricted mode, measurements were made, only after the TK450 chamber reached the requested temperature. In the continuous mode, the sample was heated at 10° C./minute and fast scans were measured as the temperature changed. After the requested temperature was reached, the sample was cooled at 35° C./minute and a slow scan measured 25° C. The temperatures chosen were based on DSC results. For the diffractometer set-up a 10 mm beam mask, 0.04 radian Soller slits and fixed (¼°) divergence and anti-scatter (⅛°) slits were inserted on the incident beam side. A fixed 0.10 mm receiving slit, 0.04 radian Soller slits and a 0.02 mm Nickel filter were inserted on the diffracted beam side. The slow scans were collected from ca. 3 to 30° 2θ with a 0.0080° step size and 100.97 sec counting time which resulted in a scan rate of approximately 0.5°/min. The fast scans were collected from ca. 3 to 30° 2θ with a 0.0167° step size and 1.905 sec counting time which resulted in a scan rate of approximately 44°/min.

### III. Single Crystals

The crystals chosen were coated with paratone oil and flash frozen on an Oxford diffraction CCD diffractometer (Oxford Instruments Xcalibur3 diffractometer equipped with a Sapphire detector). Data were collected with standard area detector techniques. The structures were solved and refined with the SHELXTL package. A standard Reitveld refinement using default parameters was calculated to obtain a room temperature cell dimensions and to check the fit of the calculated pattern from the single crystal model against the measured XRPD pattern. An overlay comparing observed and calculated X-ray powder diffraction data for Form A1 of compound 1 is provided in FIG. 17. A view of a molecule of Compound 1, in crystal Form A1, as determined by single crystal X-Ray diffraction is provided in FIG. 18. A view of the molecular packing for Form A1 is provided in FIG. 19.

### IV. Differential Scanning Calorimetry (DSC)

Thermal curves were acquired using a Perkin-Elmer Sapphire DSC unit equipped with an autosampler running Pyris software version 6.0 calibrated with Indium prior to analysis. Solid samples of 1-11 mg were weighed into 20 μL aluminum open samples pans. The DSC cell was then purged with nitrogen and heated from 0° to 275° C. at 10° C./min.

### V. Thermogravimetric (TGA)

Thermal curves were acquired using a Perkin-Elmer Pyris 1 TGA unit running Pyris software version 6.0 calibrated with calcium oxalate monohydrate. TGA samples between 1-15 mg were monitored for percent weight loss as heated from 25° to 400° C. at 10° C./min in a furnace purged with Helium at ca. 50 mL/min.

### VI. Gravimetric Vapor Sorption (GVS)

Gravimetric Vapor Sorption experiments have been carried out using the DVS-HT instrument (Surface Measurement Systems, London, UK). This instrument measures the uptake and loss of vapor gravimetrically using a recording ultra-microbalance with a mass resolution of ±0.1 μg. The vapor partial pressure (±1.0%) around the sample is controlled by mixing saturated and dry carrier gas streams using electronic mass flow controllers. The desired temperature is maintained at ±0.1° C. The samples (1-10 mg) were placed into the DVS-HT instrument at the desired temperature.

The sample was initially dried in stream of dry air (<0.1% relative humidity) for 20 hours to establish a dry mass and exposed to two 0-90% RH cycles (in 10% RH increments).

### VII. Identity, Assay, and Purity

Typically 10 μL aliquots of the sample solutions were diluted to 1 mL with acetonitrile and the assay concentrations were determined from an average of duplicate injections using the following HPLC method. The purity and impurity analyses are done using conventional HPLC.

| Column | Zorbax SB Phenyl | |
|---|---|---|
| | 150 × 4.6 mm, 3.5 μm | |
| Column temperature | 25° | C. |
| Injection volume | 5 | mL |
| Detection | 270 | nm |
| Flow rate | 1.0 | mL/min |
| Run time | 25 | min |
| Mobile phase A | 0.05% | TFA in Water |
| Mobile phase B | 0.05% | TFA in ACN |

Gradient:

| Time (min) | % A | % B |
|---|---|---|
| 0 | 80 | 20 |
| 20 | 20 | 80 |
| 20.1 | 80 | 20 |
| 25 | 80 | 20 |

### VIII. Fourier Transform Infrared Spectrometry (FTIR)

FTIR Spectra were obtained using a Thermo Electron-Nicolet Avatar 370 DTGS instrument with the Smart Orbit ATR attachment containing a diamond crystal window. Thermo Electron Omnic™ software (version 3.1) was used to compute the spectrum from 4000 to 400 cm⁻¹ from the initial interferogram. A background scan was collected before obtaining each sample spectrum. For each sample, 32 scans were obtained at 4 cm⁻¹ spectral resolution and averaged.

### Raman Spectrometry

The Raman spectra of the sample were recorded with a FT-Raman module on a vertex 70 FTIR spectrometer (Bruker RAM II, Bruker optics, Germany). A germanium photodiode was used to record FT-Raman spectra excited by an Nd:Yag laser (suppression of fluorescence). A polystyrene standard was run prior to sample analyses. Acquisition time for each spectrum was 1 minute, with a resolution of 4 cm⁻¹ and the power of the 1064 nm laser at the sample was 50 mW.

### Example 1

### Crystallization Studies for Compound 1

Crystallization studies were performed on Compound 1 to investigate polymorphism in 24 different solvents. Solvents were selected on the basis of acceptability (ICH Class 3 and 2), and also to provide a range of dielectric constants, dipole moments and functional groups. Cooling, evaporation and anti-solvent addition were also employed to obtain different forms of Compound 1. When possible, full characterization of the product was performed on the products that were generated during the screening, e.g., X-ray powder diffraction and variable-temperature X-ray powder analysis; thermal analysis; GVS; storage at 40° C./75% RH and purity by HPLC.

### Example 1(a)

### Maturation Experiments

Mixtures (40 mg of Form A1 in 400 μL of solvent) were slurried in the 24 solvents. These mixtures were slurried for 48 hours with alternating 4 hour periods at 50° C. and 5° C. (−0.5° C./min) using the HEL Polyblock™ Unit. The crystallization experiments were carried out in glass vials (2.0 mL, 32×11.6 mm). The solid products were isolated by filtration and analyzed by XRPD and thermal analysis. Results are shown in Table 4 below.

TABLE 4
Maturation Study Results

| Solvent | XRPD Analysis |
|---|---|
| 1-4 dioxane | A1 |
| 1-butanol | A1 |
| 1-propanol | A1 |
| 2-butanone | A1 |
| 2-propanol | A1 |
| 3-pentanone | A1 |
| acetone | A1 |
| acetonitrile | A1 |
| chloroform | A1 |
| dichloromethane | A1 |
| diisopropyl ether | A1 |
| dimethyl sulfoxide | A1 |
| ethanol | A1 |
| ethyl acetate | A1 |
| heptane | A1 |
| isopropyl acetate | A1 |
| methanol | A1 |
| methyl acetate | A1 |
| methyl isobutyl ketone | A1 |
| N-N-dimethylformamide | A1 |
| N-butyl acetate | A1 |
| tetrahydrofuran | A1 |
| toluene | A1 |
| water | A1 + H4A1 |

### Example 1(b)

### Slow Cool Experiments

Approximately 40 mg of Compound 1 was slurried in each of the 24 solvents (10 volumes (40 mg in 400 μL)). The samples were heated from 20° C. to 80° C. at a rate of 4.8° C./min, and after 30 minutes were cooled at a slow rate (0.25° C./min) to a final temperature of 5° C. The resulting mixtures were then kept at that temperature for 18 h using the HEL Polyblock™ Unit. The crystallization experiments were carried out in glass vials (2.0 mL, 32×11.6 mm). The solid material from each vial was isolated by filtration and evaluated by XRPD and thermal analysis. Results are shown below in Table 5.

TABLE 5
Slow Cool Study Results

| Solvent | XRPD Analysis |
|---|---|
| 1-4 dioxane | A1 |
| 1-butanol | A1 |
| 1-propanol | A1 |
| 2-butanone | A1 |
| 2-propanol | A1 |
| 3-pentanone | A1 |
| acetone | A1 |
| acetonitrile | A1 |
| chloroform | A1 |
| dichloromethane | A1 |
| diisopropyl ether | A1 |
| dimethyl sulfoxide | A1 + *H4A1 |
| ethanol | A1 + *H4A1 |
| ethyl acetate | A1 |
| heptane | A1 |
| isopropyl acetate | A1 |
| methanol | A1 |
| methyl Acetate | A1 |
| methyl isobutyl ketone | A1 |
| N-N-dimethylacetamide | A1 + *H4A1 |
| N-butyl acetate | A1 |
| tetrahydrofuran | A1 |
| toluene | A1 + *H4A1 |
| water | A1 + *H4A1 |

(*Trace of Form H4A1)

### Example 1(c)

### Fast Cool Experiments

Approximately 40 mg of Compound 1 was slurried in each of the 24 solvents (10 volumes (40 mg in 400 μL)). The samples were heated from 20° C. to 80° C. at a rate of 4.8° C./min and after 30 minutes cooled at a fast rate (10° C./min) to a final temperature of 5° C. The resulting mixtures were then kept at that temperature for 18 h using the HEL Polyblock™ Unit. The crystallization experiments were carried out in glass vials (2.0 mL, 32×11.6 mm). Results are shown in Table 6 below.

TABLE 6
Fast Cool Study Results

| Solvent | XRPD Analysis |
|---|---|
| 1-4 dioxane | A1 |
| 1-butanol | A1 |
| 1-propanol | A1 |
| 2-butanone | A1 |
| 2-propanol | A1 |
| 3-pentanone | A1 |
| acetone | A1 |
| acetonitrile | A1 |
| chloroform | A1 |
| dichloromethane | A1 |
| diisopropyl ether | A1 |
| dimethyl sulfoxide | A1 |
| ethanol | A1 |
| ethyl acetate | A1 |
| heptane | A1 |
| isopropyl acetate | A1 |
| methanol | A1 |
| methyl acetate | A1 |
| methyl isobutyl ketone | A1 |
| N-N-dimethylformamide | A1 |
| N-butyl acetate | A1 |
| tetrahydrofuran | A1 |
| toluene | A1 |
| water | A1 |

### Example 1(d)

### Evaporation Experiments

Approximately 20 mg of Compound 1 was added to a glass vial (2.0 mL, 32×11.6 mm). The solvents listed in the table below were added in 0.5 to 1.0 mL increments followed by heating with stirring to the boiling point until dissolved. If a solution was not formed by the addition of a total of 10 mL of solvent, the mixture was syringe filtered (5μ Nylon membrane). Then, all solutions were allowed to slowly evaporate to dryness under ambient conditions. The resulting solids were analyzed by XRPD. Results are shown in Table 7 below. An overlay of XRPD analyses of amorphous forms produced by the evaporation studies in acetone, 2-butanone, methyl isobutylketone, 2-propanol, toluene, chloroform, isopropyl acetate, methyl acetate, and 3 pentanone is provided in FIG. 24. Note that the weak XRPD peak from 25° to 28° 2 theta for products of the evaporation studies in chloroform, isopropyl acetate, methyl acetate and 3-pentanone resulted from a coating of these products with Kapton film prior to XRPD analysis.

TABLE 7
Evaporation Study Results

| Solvent | XRPD Analysis |
|---|---|
| acetone | Amorphous |
| acetonitrile | A1 |
| 1-butanol | A1 |
| 2-butanone | Amorphous |
| N-butyl acetate | Amorphous |
| chloroform | Amorphous |
| dichloromethane | Amorphous |
| diisopropyl ether | No sample |
| N,N-dimethylformamide | A1 |
| dimethyl sulfoxide | A1 |
| 1,4-dioxane | Amorphous |
| ethanol | A1 |
| ethyl acetate | Amorphous |
| heptane | Amorphous |
| isopropyl acetate | Amorphous |
| methanol | A1 |
| methyl acetate | Amorphous |
| methyl isobutyl ketone | Amorphous |
| 3-pentanone | Amorphous |
| 1-propanol | A1 |
| 2-propanol | Amorphous |
| tetrahydrofuran | A1 |
| toluene | Amorphous |
| water | H4A1 + A1 |

### Example 1(e)

### Quick Cool Experiments

Samples were prepared by adding approximately 40 mg of solid material into enough solvent volume to assure saturated conditions at the boiling point of each solvent. The mixtures were cooled slightly and the still warm solution filtered through a 5μ nylon membrane filter into a pre-warmed glass vial. The resulting solutions were then re-warmed to the boiling point. The solutions were then cooled to room temperature and placed in a refrigerator (ca. 4° C.) until crystal formation appeared to reach completion by visual inspection. Each refrigerated sample was decanted and the crystals were transferred to a weighing paper and dried to constant weight under ambient laboratory conditions. Samples that were difficult to decant were centrifuged at 12000 rpm for four minutes, and the solid was isolated by suction filtration. If the quick-cool procedure did not result in any solid materials, these samples were concentrated by evaporating approximately half of the solvent volume. The solutions were again placed in the refrigerator (ca. 4° C.) and any solid material that formed was isolated by decanting or centrifugation. The XRPD results for the resulting products are provided in Table 8 below.

TABLE 8
Quick Cool Study Results

| Solvent | XRPD Analysis |
|---|---|
| acetic acid | A1 |
| 1,2-dimethoxyethane | Insufficient |
| | material |
| 1-2 dichloroethane | Insufficient |
| | material |
| 1-pentanol | A1 |
| 2-butanol | A1 |
| 2-pentanone | Insufficient |
| | material |
| acetone | Insufficient |
| | material |
| benzyl alcohol | A1 |
| butyronitrile | A1 |
| chlorobenzene | A1 |
| cyclohexane | Insufficient |
| | material |
| formamide | A1 |
| formic acid | A1 |
| isobutyl alcohol | A1 |
| isopropyl acetate | No crystallization |
| methoxybenzene | A1 + Amorphous |
| methyl tert-butyl ether | Insufficient |
| | material |
| N,N-dimethylacetamide | A1 |
| N-methylpyrrolidinone | A1 |
| tert-butanol | A1 |
| tetrahydropyran | No crystallization |
| tetrahydrothiophene | A1 + Amorphous |
| 1,1-dioxide | |
| toluene | No crystallization |

### Example 2

### Preparation and Analysis of Form A1

### Example 2(a)

### Preparation of Form A1 by Synthesis

A reactor was charged at 20° C. with 6-[4-[3-((R)-2-methylpyrrolidine-1-yl)-propoxy]phenyl]2H-pyridazine-3-one free base (1 eq or 4.43 Kg), iPrOH (15 V) and MTBE (15 V). The mixture was stirred (80 rpm) at 20° C. for 5 minutes. The mixture was then heated to 67° C. until complete dissolution, and was maintained at that temperature for 45 min. The mixture was then cooled to 50° C. and filtered through a polishing cellulose lens. At 50° C., hydrochloric acid in 2-propanol (1.2 eq) was added over 90 min via the feed vessel to the solution. The resulting slurry was cooled to 10° C. (−0.3° C./min) and a contact of 2 hr at 10° C. was maintained. The mixture was then filtered by centrifugation. The collected solid was washed with MTBE (3 V) and dried under vacuum at 50° C. overnight. The recovery of 6-[4-[3-((R)-2-methylpyrrolidine-1-yl)-propoxy]phenyl]2H-pyridazine-3-one HCl was 96.4%. XRPD analysis of Form A1 is provided in FIG. 1.

### Example 2(b)

### Preparation of Form A1 by Solid-Solid Transition

Form H4A1 (100 mg) was stored at 25° C./0% RH for 7 days. Analysis by XRPD confirmed quantitative conversion of the material to Form A1.

### Example 2(c)

### Preparation of Crystal Form A1 for Single Crystal Study

Single crystals were prepared as part of a standard evaporative crystal screen by adding 20 mg of 6-[4-[3-((R)-2-methylpyrrolidine-1-yl)-propoxy]phenyl]2H-pyridazine-3-one HCl solid material to 0.2 ml of DMSO. The solution was left standing for several days until crystals formed. The crystals were isolated and then dried in a vacuum oven to remove residual solvent.

A colorless blade of crystal Form A1, approximate dimensions 0.07 mm×0.33 mm×0.55 mm, was used for the X-ray crystallographic analysis. The X-ray intensity data were measured at 295(2) K on a Oxford Instruments Xcalibur3 diffractometer system equipped with a graphite monochromator and a MoKα fine-focus sealed tube (λ=0.71073 Å) operated at 2 kW power (50 kV, 40 mA). The detector was placed at a distance of 50 mm. from the crystal.

During the experiment, 652 frames were collected with a scan width of 1.00° in ω. All frames were collected with an exposure time of 60 sec/frame. The frames were integrated with the Oxford diffraction package CrysAlis RED. The integration of the data using a monoclinic cell yielded a total of 6856 reflections to a maximum θ angle of 21.96°, of which 2215 were independent, completeness=99.1%, Rᵢₙₜ=5.69%, R_{sig}=4.97%) and 1848 were greater than 2σ(F²). The final cell constants of a=10.8386(10) Å, b=6.9192(5) Å, c=24.432(3) Å, α=90°, β=95.092(9)°, γ=90°, volume=1825.0(3) Å3, are based upon the refinement of the XYZ-centroids of 2553 reflections above 20 σ(I) with 3.8460°<2θ<26.4995°. Analysis of the data showed negligible decay during data collection. Data were corrected with an analytical numeric absorption correction using a multifaceted crystal model as programmed in the Oxford diffraction package, CrysAlis RED. The minimum and maximum transmission corrections were 0.930 and 0.989. The calculated minimum and maximum transmission coefficients (based on crystal size) are 0.8865 and 0.9845.

The structure was solved and refined using the Bruker SHELXTL (Version 6.1) Software Package, using the space group C2, with Z=4 for the formula unit, C₁₈H₂₄N₃O₂ HCl. The final anisotropic full-matrix least-squares refinement on F2 with 226 variables converged at R1=6.72%, for the observed data and wR2=17.79% for all data. The goodness-of-fit was 1.449. The largest peak on the final difference electron density synthesis was 0.236 e⁻/Å³ and the largest hole was −0.247 e⁻/Å³ with an RMS deviation of 0.061 e⁻/Å³. On the basis of the final model, the calculated density was 1.277 g/cm³ and F(000), 748 e⁻.

In order to check the consistency of the single crystal model with its unit cell at room temperature and the measured powder pattern, the single crystal cell constants were refined in a default Rietveld refinement against the powder data. Single crystal and powder values are:

| Single crystal | Rietveld powder |
|---|---|
| a = 10.8386(10) Å | 10.833(3) Å |
| b = 6.9192(5) Å | 6.916(2) Å |
| c = 24.432(3) Å | 24.447(8) Å |
| β = 95.092(9)° | 95.08(2)° |

### Example 2(d)

### Characterization of Form A1 by Variable Temperature XRPD (VT-XRPD)

A variable temperature study was performed for Form A1 according to the protocol set out in II above. No solid-solid transformation was observed in the temperature range of 20° C. to 250° C. for Form A1 (No evidence of the polymorphic transformation of Form A1 to Form H4A1 was observed). An overlay showing data collected in the Variable Temperature XRPD analysis of Form A1 is provided in FIG. 2.

### Example 2(e)

### Characterization of Form A1 by Thermal Analysis

Differential Scanning calorimetry and Thermogravimetric analysis for Form A1 were carried out according to the protocol set out in part V above. Form A1 shows a single peak at ca. 242° C. with an enthalpy of fusion (ΔHFus) of 113.9 J/g. No loss of mass is detected by TGA. The existence of a desolvation process was discounted because no loss of weight was detected by TGA. An overlay of the DSC and TGA analyses for Form A1 is provided in FIG. 3.

### Example 2(f)

### Characterization of Form A1 by Water Sorption

The amount of moisture adsorbed by Form A1 was less than 0.8% and increased to approximately 1.8% at 90% RH. The adsorption and desorption curves overlap suggesting that Form A1 is not hygroscopic and did not appear to form a hydrate, under these experimental conditions. The GVS isotherm plot for Form AS1 is provided in FIG. 4. An overlay of XRPD analyses before and after GVS is provided in FIG. 5, showing no significant changes after GVS.

### Example 3

### Preparation and Analysis of Form B1

### Example 3(a)

### Preparation of Form B1 by Desolvation

Form H4A1 (12 mg) was heated to 100° C. under nitrogen flow in an Anton Paar TK450 camera. Analysis by XRPD confirmed quantitative conversion of the material to Form B1. An X-ray powder diffractogram for Form B1 is depicted herein in FIG. 8.

### Example 3(b)

### Characterization of Form B1 by VT-XRPD

By dehydrating Form H4A1 in a temperature interval of 25-100° C. at 0% RH, evidence of the polymorphic transformation of Form B1 to Form A1 was observed at 220° C. An overlay showing data collected in the Variable Temperature XRPD analysis of Form B1 is provided in FIG. 9.

### Example 3(c)

### Characterization of Form B1 by Thermal Analysis

A thermal curve was acquired (Differential Scanning calorimetry and Thermogravimetric analysis) for Form B1 according to the protocol set out in part V above. The DSC curve of anhydrate B1 exhibited an exotherm attributed to the solid-solid transformation from Form B1 to Form A1. The heat of transition for B1 to A1, estimated from the exotherm on the DSC curve, was −4.50 J/g. An overlay of the DSC and TGA analyses for Form B1 is provided in FIG. 10.

### Example 4

### Preparation and Analysis of Form H4A1

### Example 4(a)

### Preparation of Form H4A1 by Recrystallization of Form A1 from Water

Approximately 79.5 mg of Form A1 was added in 1.2 mL of water. The sample was warmed to 40° C. to give a clear solution. The solution was then allowed to evaporate without stirring in a fume hood over three days. Analysis by XRPD confirmed that the collected product was Form B1. The recovery was 84%. An X-ray powder diffractogram for Form H4A1 is depicted herein in FIG. 11.

### Example 4(b)

### Preparation by Solid-Solid Transition

Approximately 100 mg of Form A1 was exposed to 100% RH at 25° C. over 1 day. Analysis by XRPD confirmed quantitative conversion of the material to Form H4A1.

### Example 4(c)

### Characterization of Form H4A1 by Thermal Analysis

Differential Scanning calorimetry and Thermogravimetric analysis for Form H4A1 were carried out according to the protocol set out in part V above. The DSC thermograms of Form H4A1 show the presence of different endothermic peaks depending on the experimental conditions. In an open pan, Form H4A1 exhibits a broad endothermic peak from approximately 0 to 100° C., corresponding to the total amount of water that escapes from the crystal. These endothermic events correspond to the dehydration process involving the escape of water from the lattice. Desolvation occurs in the solid state with an endothermic peak. The observed exothermic transition is due to the crystallization of the solvent-free form from the melt. Then the melting peak of the solvent-free form is observed. Form H4A1 in TGA experiments loses an average weight of 16.2% between 20 and 100° C. The theoretical value for incorporation of four moles of water with one mole of 6-[4-[3-((R)-2-methylpyrrolidine-1-yl)-propoxy]phenyl]2H-pyridazine-3-one hydrochloride HCl is 17.1%. An overlay of the DSC and TGA analyses for Form H4A1 is provided in FIG. 12.

### Example 4(d)

### Characterization of Form H4A1 by Water Sorption—GVS (70-0-90% RH)

FIG. 13 displays the dynamic vapor sorption data collected on Form H4A1 (3 cycles). The DVS experiment was started at 70% RH (red line) to ensure that there was no moisture loss. The sample was kept at 70% RH for 2 hours. From 80-90% RH there is a significant uptake suggesting bulk absorption (hysteresis gap). After each cycle the moisture uptake decreases to 21, 15, and 2.3% respectively at 90% RH. Significant polymorphic changes were observed by XRPD analysis of the “after GVS” samples. FIG. 14 shows a mixture of Forms A1 and H4A1 for the first and second cycle. Complete conversion of Form H4A1 to Form A1 was observed at the third cycle.

### Example 4(e)

### FTIR and FT-Raman Method for Identification Assay (Forms A1 and H4A1)

Comparison of the FTIR and Raman spectra for Form A1 (FIGS. 6 and 7) with the FTIR and Raman spectra for Form H4A1 (FIGS. 15 and 16) show differences in the carbonyl stretching region for the FTIR. In the FTIR the absorption values at 2549 and 2646 cm⁻¹ confirm the presence of the HCl salt. For Form H4A1, the IR spectrum shows a large water peak at ˜3300 cm⁻¹. The peaks at 2700 and 2616 cm⁻¹ are shifted about 150 cm⁻¹ from the same peaks in Form A1. Moreover, the CO and CN stretches are shifted also, but only ˜15 cm-1 for Form H4A1. Table 9 below summarizes these observed differences.

TABLE 9

| FTIR | FTIR comparison | | Raman comparison | |
|---|---|---|---|---|
| comparison | C═O cm⁻¹ | CN cm⁻¹ | C═O cm⁻¹ | CN cm⁻¹ |
| Form A1 | 1669 | 1174 | 1612 | 1178 |
| Form H4A1 | 1665 | 1181 | 1601 | 1187 |

### Example 4(f)

### Single Crystal (Form A1) Structure Determination of Compound 1

Table 10 below lists sample and crystal data for the single crystal structure determination of crystal form A1.

TABLE 10
Data for crystal structure determination of Form A1

| Crystallization solvents | dimethyl sulfoxide | |
|---|---|---|
| Crystallization method | slow evaporation | |
| Chemical formula | C₁₈H₂₄N₃O₂ HCl | |
| Empirical formula | C₁₈H₂₅N₃O₂Cl | |
| Formula weight | 350.86 | |
| Temperature | 295(2) K | |
| Wavelength | 0.71073 Å | |
| Crystal size | 0.55 × 0.33 × 0.07 mm | |
| Crystal habit | Colorless blade | |
| Crystal system | Monoclinic | |
| Space group | C₂ | |
| Unit cell dimensions | a = 10.8386(10) Å | α = 90° |
| | b = 6.9192(5) Å | β = 95.092(9)° |
| | c = 24.432(3) Å | γ = 90° |

| | |
|---|---|
| Volume | 1825.0(3) Å³ |
| Z | 4 |
| Density (calculated) | 1.277 Mg/m³ |
| Absorption coefficient | 0.225 mm⁻¹ |
| F(000) | 748 |

Table 11 below lists information for the data collection and structure refinement for crystal structure determination of crystal form A1.

TABLE 11
Data Collection and Structure Refinement
for Form A1 of Compound 1.

| | |
|---|---|
| Diffractometer | Oxford Instruments Xcalibur3 |
| | diffractometer |
| Radiation source | fine-focus sealed tube, MoKα |
| Generator power | 2 kW (50 kV, 40 mA) |
| Detector distance | 50 |
| Data collection method | omega scans |
| Theta range for data collection | 3.94 to 21.96° |
| Index ranges | −11 ≦ h ≦ 11, −7 ≦ k ≦ |
| | 7, −25 ≦ l ≦ 25 |
| Reflections collected | 6856 |
| Independent reflections | 2215 [R(int) = 0.0569] |
| Coverage of independent | 99.1% |
| reflections | |
| Variation in check reflections | n/a |
| Absorption correction | analytical |
| Max. and min. transmission | 0.9845 and 0.8865 |
| Structure solution technique | direct methods |
| Structure solution program | SHELXS-97 (Sheldrick, 1990) |
| Refinement technique | full-matrix least-squares on F² |
| Refinement program | SHELXL-97 (Sheldrick, 1997) |
| Function minimized | Σ w(Fₒ² − F_{c}²)² |
| Data/restraints/parameters | 2215/3/226 |
| Goodness-of-fit on F² | 1.448 |
| Δ/σₘₐₓ | 0.007 |
| Final R indices(*) | |
| 1989 data; I > 2s (I) | R¹ = 0.0672, wR² = 0.1701 |
| all data | R¹ = 0.0826, wR² = 0.1779 |
| Weighting scheme | w = 1/[σ²(Fₒ²) + [(0.08P)²] |
| where P = [MAX(Fₒ², 0) + 2F_{c}²]/3 | |
| Absolute structure parameter | −0.06(16) |
| Largest diff. peak and hole | 0.236 and −0.247 eÅ⁻³ |
| R.M.S. deviation from the mean | 0.061 eÅ⁻³ |

R = Σ |Fₒ − F_{c}|/Σ |Fₒ| & wR2 = Σ w(Fₒ² − F_{c}²)²/Σ (Fₒ²)²

Table 12 below lists the atomic coordinates and equivalent isotropic atomic displacement parameters (Å2) for the crystal structure determination of crystal form A1. (Å2) U(eq) is defined as one third of the trace of the orthogonalized Uᵢⱼ tensor.

TABLE 12

| | x/a | y/b | z/c | U(eq) |
|---|---|---|---|---|
| Cl1 | −0.27422(15) | 0.9177(2) | 0.12853(8) | 0.0668(6) |
| O1 | 0.7455(7) | −0.1300(13) | 0.4999(3) | 0.145(3) |
| O2 | 0.0317(4) | 0.2887(6) | 0.26623(18) | 0.0583(13) |
| N1 | 0.6196(6) | 0.0757(11) | 0.4565(3) | 0.080(2) |
| N2 | 0.5150(6) | 0.1322(11) | 0.4237(3) | 0.078(2) |
| N3 | −0.2247(4) | 0.4815(6) | 0.13248(18) | 0.0378(12) |
| C1 | 0.6505(8) | −0.1059(19) | 0.4677(3) | 0.096(3) |
| C2 | 0.5680(9) | −0.2533(16) | 0.4432(3) | 0.102(4) |
| C3 | 0.4669(8) | −0.2007(12) | 0.4124(3) | 0.085(3) |
| C4 | 0.4437(7) | −0.0024(12) | 0.4026(2) | 0.0563(19) |
| C5 | 0.3318(6) | 0.0763(9) | 0.3669(2) | 0.0453(16) |
| C6 | 0.2321(5) | −0.0391(9) | 0.3481(2) | 0.0474(17) |
| C7 | 0.1382(6) | 0.0337(9) | 0.3150(3) | 0.0516(18) |
| C8 | 0.1341(5) | 0.2242(9) | 0.2988(2) | 0.0463(17) |
| C9 | 0.2361(6) | 0.3409(10) | 0.3155(3) | 0.062(2) |
| C10 | 0.3298(6) | 0.2649(11) | 0.3488(3) | 0.064(2) |
| C11 | 0.0277(7) | 0.4860(10) | 0.2506(3) | 0.0578(19) |
| C12 | −0.0949(6) | 0.5266(9) | 0.2199(3) | 0.0559(18) |
| C13 | −0.1123(5) | 0.4240(11) | 0.1659(2) | 0.0456(14) |
| C14 | −0.3439(5) | 0.4327(11) | 0.1549(3) | 0.0626(18) |
| C15 | −0.4371(7) | 0.428(2) | 0.1064(4) | 0.118(4) |
| C16 | −0.3735(7) | 0.4387(14) | 0.0579(3) | 0.080(2) |
| C17 | −0.2359(5) | 0.4016(10) | 0.0743(2) | 0.0460(15) |
| C18 | −0.1445(7) | 0.4989(10) | 0.0394(3) | 0.070(2) |

### Example 5

### Stability of Solid State Forms A1 and H4A1

### Solid State Stress Stability

Stress stability studies were performed to assess the influence of temperature and humidity on stability of Forms A1 and H4A1. A stability-indicating HPLC assay method was developed for quantification of Compound 1. The developed method, described above in section VII, is specific, accurate, precise and robust.

### Example 5(a)

### Form A1 Stability at 40° C./75% RH

In the solid state, Form A1 was not observed to take up water from the environment at standard ICH stressed conditions of 40° C./75% RH after 4 weeks. In addition, chemical degradation was not observed in Form A1 under these stressed conditions (data provided in Table 13 below).

TABLE 13
Stability Data for Form A₁ at 40° C./75% RH

| Days | XRPD | Area Purity (%) |
|---|---|---|
| 0 | A1 | 99.5 |
| 7 | A1 | 99.6 |
| 14 | A1 | 99.7 |
| 28 | A1 | 99.5 |

### Example 5(b)

### Form A1 Stability at 40° C./75% RH

Form H4A1 was observed to be physically and chemically stable for 28 days when stored at 40° C. and 75% RH (data provided in Table 14 below).

TABLE 14
Stability Data for Form H₄A₁ at 40° C./75% RH

| Days | XRPD | Area Purity (%) |
|---|---|---|
| 0 | H4A1 | 99.6 |
| 7 | H4A1 | 99.5 |
| 14 | H4A1 | 99.5 |
| 28 | H4A1 | 99.5 |

### Example 5(c)

### Stability of Forms A1 and H4A1 at Different Humidity Conditions at Room Temperature

Approximately 10 mg of Forms A1 and H4A1 were stored in closed desiccators with saturated solutions of various salts resulting in relative humidity conditions as listed in the tabulated data below. Samples were analyzed by XRPD at 3 days, 1 and 4 weeks. Under high humidity conditions (˜85% RH) conversion of Form A1 to Form H4A1 was observed (3 days), Conversion of Form H4A1 to Form A1 was observed at 43% RH (1 week).

TABLE 15
XRPD Analysis of Stability Study Samples of Form H4A1

| | | XRPD analysis | | |
|---|---|---|---|---|
| Relative | | After | After | After |
| Humidity (% RH) | | 3 days | 1 week | 4 weeks |
| 0 | (phosphorus pentoxide) | A1 + H4A1 | A1 | A1 |
| 11.3 | (lithium chloride) | H4A1 | A1 | A1 |
| 43.1 | (potassium carbonate) | H4A1 | A1 | A1 |
| 75.3 | (sodium chloride) | H4A1 | H4A1 | H4A1 |

TABLE 16
XRPD Analysis of Stability Study Samples of Form A1

XRPD analysis

| | | | |
|---|---|---|---|
| Relative | After | After | After |
| Humidity (% RH) | 3 days | 1 week | 4 weeks |

| | | | | |
|---|---|---|---|---|
| 0 | (phosphorus pentoxide) | A1 | A1 | A1 |
| 11.3 | (lithium chloride) | A1 | A1 | A1 |
| 43.1 | (potassium carbonate) | A1 | A1 | A1 |
| 75.3 | (sodium chloride) | A1 | A1 | A1 |
| 85.1 | (potassium chloride) | H4A1 | H4A1 | H4A1 |
| 93.5 | (potassium nitrate) | H4A1 | H4A1 | H4A1 |
| 97.6 | (potassium sulfate) | H4A1 | H4A1 | H4A1 |
| 100 | (water) | H4A1 | H4A1 | H4A1 |

### Example 5(d)

### Stability of Forms A1 and H4A1 to Mechanical Stress (Grinding)

A Wig-1-Bug (Piketech, USA) was used to grind Forms A1 and H-4A1. Each sample (50 mg) was ground for periods of 5, 10, 15 and 30 minutes. Each grinding was carried out in a 2.82 cm³ container using 0.9 g stainless steel ball (0.6 mm diameter). The vial was swung through a 6.5° arc at 3200 rpm, causing the ball to strike the end of the vial at over 100 Hz.

### Form A1 Stability to Mechanical Stress

After thirty minutes of grinding, the XRPD patterns showed that crystallinity had been significantly reduced. Nonetheless, as the remaining peaks were in the same position as the starting material, the grinding did not generate a change in crystal form. An overlay showing results of this mechanical stress assessment is provided in FIG. 22.

### Form H4A1 Stability to Mechanical Stress

After ten minutes of grinding, the XRPD pattern for ground Form H4A1 is similar to the pattern for ground A1 and H4A1. An overlay showing results of this mechanical stress assessment is provided in FIG. 23.

1. Crystalline form A1 of Compound 1: [chemical structure]
characterized by an X-ray powder diffraction pattern having any selection of from five to ten X-ray powder diffraction peaks selected from 3.75, 10.98, 14.62, 15.25, 15.55, 15.88, 16.48, 16.64, 17.19, 18.26, 20.63, 21.08, 21.67, 23.02, 23.29, 23.56, 24.43, 25.78, 26.07, 26.28, 26.33, 27.42, 27.95, 28.40, 29.35, and 29.77 degrees two theta±0.2 degrees two theta.

2. The crystalline Form A1 of Compound 1, according to claim 1, characterized by an X-ray powder diffraction pattern having peaks at 3.75, 10.98, 14.62, 15.25 and 15.88 degrees two theta±0.2 degrees two theta.

3. The crystalline Form A1 of Compound 1, according to claim 2, further characterized by one or more additional X-ray powder diffraction peaks selected from 16.48, 16.64, 17.19, 18.26 and 20.63 degrees two theta±0.2 degrees two theta.

4. The crystalline Form A1 of Compound 1, according to claim 2, further characterized by additional data selected from one or more of: a powder X-ray diffraction pattern as depicted in FIG. 1, a DSC curve having an endotherm with an onset at 239.5 degrees C. (ΔH 113.9 J/g), a DSC curve as depicted in FIG. 2, A TGA curve as depicted in FIG. 2, an FTIR spectrum as depicted in FIG. 6, and a Raman spectrum as depicted in FIG. 7.

5. The crystalline Form A1 of Compound 1, according to claim 2, further characterized by a single crystal structure in a C2 space group with unit cell dimensions of: a=10.8386(10) Å, b=6.9192(5) Å, c=24.432(3) Å, α=γ=90°, β=95.092(9)° and Volume=1825.0(3) Å³.

6. Crystalline form H4A1 of Compound 1: [chemical structure]
characterized by an X-ray powder diffraction pattern having any selection of from five to eight X-ray powder diffraction peaks selected from 5.72, 11.40, 12.95, 16.45, 17.11, 17.34, 21.45, and 22.26 degrees two theta±0.2 degrees two theta.

7. The crystalline Form H4A1 of Compound 1, according to claim 6, characterized by an X-ray powder diffraction pattern having peaks at 5.72, 11.40, 12.95, 16.45 and 17.11 degrees two theta±0.2 degrees two theta.

8. The crystalline Form H4A1 of Compound 1, according to claim 7, further characterized by one or more additional X-ray powder diffraction peaks selected from 17.34, 21.45, and 22.26 degrees two theta±0.2 degrees two theta.

9. The crystalline Form H4A1 of Compound 1, according to claim 7, further characterized additional data selected from one or more of: a powder X-ray diffraction pattern as depicted in FIG. 11, a broad endotherm at 58° C., a DSC curve as depicted in FIG. 12, a TGA weight loss over the temperature range of 25-150° C. of 16.2, a TGA curve as depicted in FIG. 12, an FTIR spectrum as depicted in FIG. 15, and a Raman spectrum as depicted in FIG. 16.

10. Crystalline form B1 of Compound 1: [chemical structure]
characterized by an X-ray powder diffraction pattern having peaks at 6.87, 13.79, 15.76, 19.25 and 25.79 degrees two theta±0.2 degrees two theta.

11. The crystalline Form B1 of Compound 1, according to claim 10, further characterized additional data selected from one or more of: a powder X-ray diffraction pattern as depicted in FIG. 8, a DSC curve as depicted in FIG. 10, and a TGA curve as depicted in FIG. 10.

12. A pharmaceutical composition comprising the crystalline form A1 of Compound 1 according to claim 1 and at least one pharmaceutically acceptable excipient.

13. A pharmaceutical composition comprising the crystalline form H4A1 of Compound 1 according to claim 6 and at least one pharmaceutically acceptable excipient.

14. A pharmaceutical composition comprising the crystalline form B1 of Compound 1 according to claim 10 and at least one pharmaceutically acceptable excipient.

15. A method for the treatment of a disorder mediated by a histamine H3 receptor in a human subject in need of such treatment, the method comprising administering an effective amount of a composition according to claim 12.

16. The method according to claim 15, wherein the disorder is selected from: narcolepsy or sleep/wake disorders, feeding behavior, eating disorders, obesity, cognition, arousal, memory, mood disorders, mood attention alteration, attention deficit hyperactivity disorder, Alzheimer's disease/dementia, schizophrenia, pain, stress, migraine, motion sickness, depression, psychiatric disorders, epilepsy, gastrointestinal disorders, respiratory disorders, inflammation, and myocardial infarction.

17. A method for the treatment of a disorder mediated by a histamine H3 receptor in a human subject in need of such treatment, the method comprising administering an effective amount of a composition according to claim 13.

18. The method according to claim 17, wherein the disorder is selected from: narcolepsy or sleep/wake disorders, feeding behavior, eating disorders, obesity, cognition, arousal, memory, mood disorders, mood attention alteration, attention deficit hyperactivity disorder, Alzheimer's disease/dementia, schizophrenia, pain, stress, migraine, motion sickness, depression, psychiatric disorders, epilepsy, gastrointestinal disorders, respiratory disorders, inflammation, and myocardial infarction.

19. A method for the treatment of a disorder mediated by a histamine H3 receptor in a human subject in need of such treatment, the method comprising administering an effective amount of a composition according to claim 14.

20. The method according to claim 19, wherein the disorder is selected from: narcolepsy or sleep/wake disorders, feeding behavior, eating disorders, obesity, cognition, arousal, memory, mood disorders, mood attention alteration, attention deficit hyperactivity disorder, Alzheimer's disease/dementia, schizophrenia, pain, stress, migraine, motion sickness, depression, psychiatric disorders, epilepsy, gastrointestinal disorders, respiratory disorders, inflammation, and myocardial infarction.
